(19) United States
(12) Patent Application Publication
(10) Pub. No.: US 2023/0329619 A1
(43) Pub. Date: Oct. 19, 2023

(54) WIRE ORGANIZATION SYSTEM FOR ECG MACHINE

(71) Applicant: Melvina Smith Roberts, Evans, GA (US)

(72) Inventors: Melvina Smith Roberts, Evans, GA (US); Eric Cotton, Stratford, CT (US)

(21) Appl. No.: 18/299,678

(22) Filed: Apr. 12, 2023

Related U.S. Application Data
(60) Provisional application No. 63/330,801, filed on Apr. 14, 2022.

(51) Int. Cl. A61B 5/282; A61B 5/30

(52) U.S. Cl. CPC A61B 5/303; A61B 5/282

(57) ABSTRACT

A cable organizer includes an outer housing, an axle extending longitudinally through the outer housing, and a plurality of spools supported on the axle such that the plurality of spools is configured to independently rotate relative to one another about a longitudinal axis defined by the axle. The spools are configured to let out a respective ECG cable when an end of the ECG cable is pulled.

### CROSS-REFERENCE TO RELATED APPLICATIONS

This application claims the benefit of and priority to U.S. Provisional Pat. Application No. 63/330,801 filed on Apr. 14, 2022, the entire contents of which are incorporated by reference herein.

### BACKGROUND

A heart rate monitor or an electrocardiogram is typically used to record the electrical signals of a person’s heart. It’s a common and painless test used to quickly detect heart problems and monitor heart health. Electrocardiograms, also called ECGs or EKGs, may be done in a doctor’s office, a clinic or a hospital room. ECG machines are standard equipment in operating rooms and ambulances. They typically include an ECG sampling box, a software program, one or more ECG cables (configured as banana connectors or the like), a plurality of leads or wires, and clips or surfaces that connect to the person’s body surface.

Use of portable heart rate monitors may be complicated due to tangling and disorganization of wires associated with the monitor. The wires of a typical heart rate monitor are long and may become tangled. A rubber band is sometimes used in an attempt to shorten the length of the wires. This rubber band method fails to organize the wires, and when the rubber band is removed, the wires remain unorganized and may become tangled shortly thereafter. A back of a portable heart rate monitor may be equipped with lead labels provided to help a user associate each wire with its appropriate position on the body of a patient. Alternate methods include simply wrapping the wires around each other, and placing the portable heart rate monitor onto a surface or in a holding device.

Some ECG systems include a holder for the portable heart rate monitor. The holder may be associated with a screen that shows the results of the ECG and a surface, such as a bed, upon which the patient will lie. A further fastening clip may be provided that is used to secure the wires together. The fastening clip is rigid and includes slots within which each wire can be positioned. A right fastening clip and a left fastening clip may be utilized to organize the wires.

In use of a typical portable heart rate monitor, the wires may be strewn over a body surface of a patient, and the patient’s body surface (ex: an arm) may easily become entangled with the wires and detach the contact surface of the wires from the patient - resulting in a loss of ECG reading.

A typical issue with ECG machines is that the wires or leads often become tangled. In emergency situations where a patient needs care immediately, it is inconvenient to untangle these wires. Therefore, there is a need for a wire management or organization system for ECG machines.

### BRIEF SUMMARY

According to an aspect of the present disclosure, a cable organizer is provided that includes an outer housing, an axle extending longitudinally through the outer housing, and a plurality of spools supported on the axle such that the plurality of spools is configured to independently rotate relative to one another about a longitudinal axis defined by the axle. Each spool defines a channel therethrough configured for passage of a respective electrocardiogram (ECG) cable.

According to another aspect of the present disclosure, a cable organizer is provided that includes a plurality of electrocardiogram (ECG) cables, an outer housing defining an inner chamber therein, an axle extending longitudinally through the outer housing and supported by the outer housing, and a plurality of spools supported on the axle. A first ECG electrode of the plurality of ECG electrodes has a first end, a second end, and a body portion extending between the first and second ends. A first spool of the plurality of spools defines a channel therethrough configured for passage of the body portion of the first ECG cable. The first spool is resiliently biased toward a wound condition in which the body portion of the first ECG cable is wound about the first spool. The first spool is configured to rotate from the wound condition to unwind the body portion of the first ECG cable when a pulling force is applied to the first or second end of the ECG cable.

### BRIEF DESCRIPTION OF THE SEVERAL VIEWS OF THE DRAWINGS

A more particular description will be rendered by reference to exemplary embodiments that are illustrated in the accompanying figures. Understanding that these drawings depict exemplary embodiments and do not limit the scope of this disclosure, the exemplary embodiments will be described and explained with additional specificity and detail through the use of the accompanying drawings in which:

FIG. 1A is a first side, perspective view illustrating a cable organizer connected to cables of a portable heart rate monitor, according to an embodiment of the present disclosure;

FIG. 1B is a second side, perspective view illustrating the cable organizer of FIG. 1A;

FIG. 2 is a perspective view, with parts separated, of a spool assembly disassembled from an outer housing of the cable organizer of FIG. 1A;

FIG. 3 is a plan view, with parts separated, of a spool of the spool assembly of FIG. 2;

FIG. 4 is a transverse cross-sectional view of the cable organizer of FIG. 1A;

FIG. 5 is another transverse cross-sectional view of the cable organizer of FIG. 1A

FIG. 6 is a perspective view illustrating a cable organizer connected to cables of a portable heart monitor, according to another embodiment of the present disclosure;

FIG. 7 is a plan view, with parts separated, illustrating a spool of the cable organizer of FIG. 6

FIG. 8A is a perspective view, with parts separated, of the spool of FIG. 7; and

FIG. 8B is a perspective view of the spool of FIG. 8A in an assembled state.

Various features, aspects, and advantages of the exemplary embodiments will become more apparent from the following detailed description, along with the accompanying drawings in which like numerals represent like components throughout the figures and detailed description. The various described features are not necessarily drawn to scale in the drawings but are drawn to aid in understanding the features of the exemplary embodiments.

The headings used herein are for organizational purposes only and are not meant to limit the scope of the disclosure or the claims. To facilitate understanding, reference numerals have been used, where possible, to designate like elements common to the figures.

### DETAILED DESCRIPTION

Reference will now be made in detail to various exemplary embodiments. Each example is provided by way of explanation and is not meant as a limitation and does not constitute a definition of all possible embodiments. It is understood that reference to a particular “exemplary embodiment” of, e.g., a structure, assembly, component, configuration, method, etc. includes exemplary embodiments of, e.g., the associated features, subcomponents, method steps, etc. forming a part of the “exemplary embodiment.”

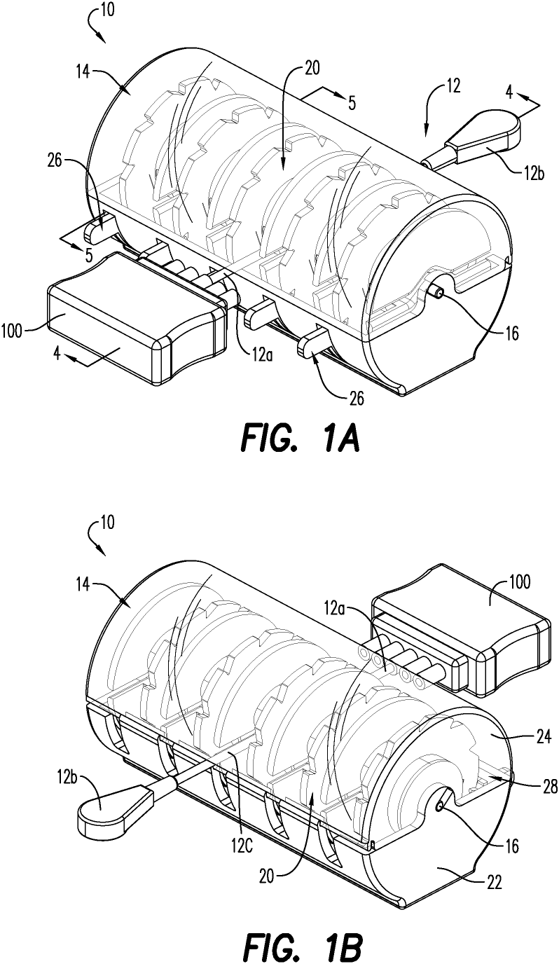

With reference to FIG. 1A-5, a cable organizing system or cable organizer 10 and associated components thereof are illustrated. In aspects, the cable organizer 10 may be configured to organize any suitable cable, such as an ECG cable 12, wire, cord, lead, or the like. The ECG cables 12 may be ECG electrodes configured to couple to a portable heart rate monitor 100. Each of the ECG cables 12 includes a first end 12a configured to couple to the portable heart rate monitor 100, a second end 12b configured to couple to a patient, and a body portion 12c extending between the first and second ends 12a, 12b. Only a single cable 12 is illustrated, however it is contemplated that the cable organizer 10 may be configured to accommodate any suitable number of cables, such as, for example, two, three, four, five, or more. It is contemplated that the cables 12 may be longer than illustrated in the figures.

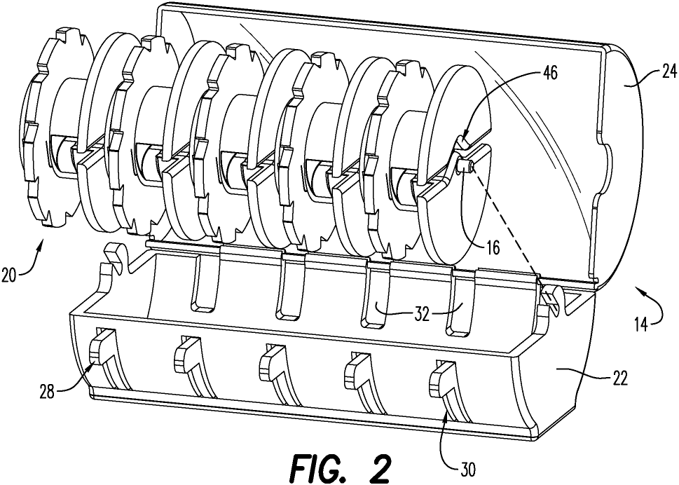

The cable organizer 10 generally includes an outer housing 14, an axle 16 detachably coupled to the outer housing 14, and a plurality of spools 20 supported on the axle 16. With reference to FIGS. 1A, 1B, and 2, the outer housing 14 of the cable organizer 10 includes a base 22, a cover 24 pivotably coupled to the base 22, and a plurality of pawls 26 pivotably coupled to the base 22. The base 22 and the cover 24, when in a closed state (as shown in FIGS. 1A and 1B), define an inner chamber 28 of the outer housing 14 for enclosing the plurality of spools 20 therein. The base 22 defines a first series of openings 30 in a first side of the base 22, and a second series of openings 32 in a second side of the base 22. Each of the first openings 30 are aligned with a respective second opening 30 to allow for a passage of the cables 12 through the outer housing 14. The axle 16 extends longitudinally through the outer housing 14 and is supported (e.g., rotationally) by the base 22 of the outer housing 14. The axle 16, with the spools 20 attached thereto, is removable from the outer housing 14 to allow for cleaning and/or replacement of the ECG cables 12.

With reference to FIGS. 2-5, the spools 20 are supported on the axle 16 and are each configured to rotate relative to and about the axle 16. The plurality of spools 20 are configured to slide off the axle 16 when the axle 16 is removed from the outer housing 14 to allow for cleaning of the cable organizer 10 and/or replacement of the ECG cables 12. Since the plurality of spools 20 are identical or substantially similar to one another, only details of a first spool 20 will be provided herein.

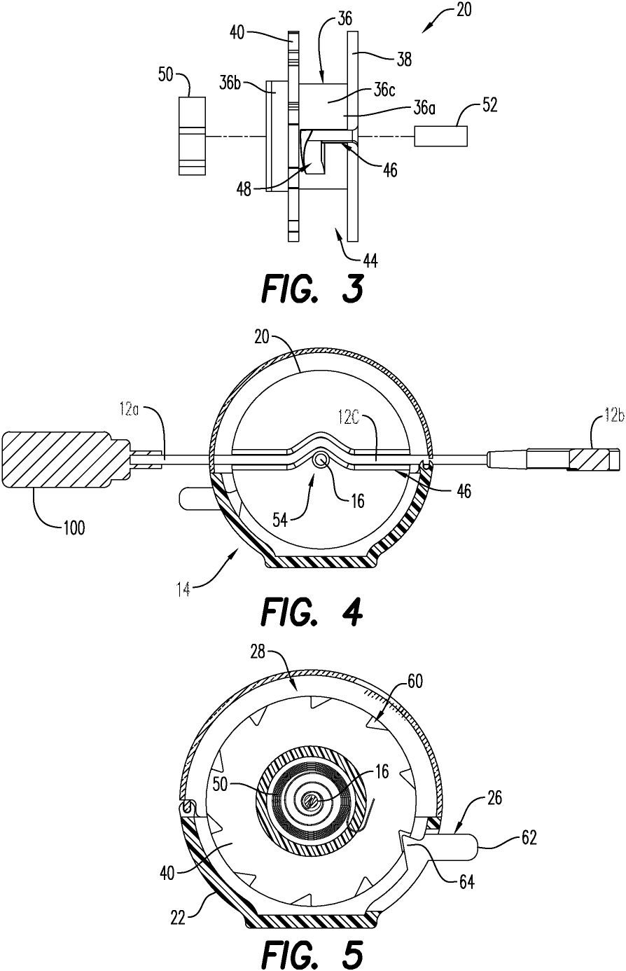

As illustrated in FIG. 3, for example, the first spool includes a central hub 36, a plate 38, and a ratchet wheel 40. The central hub 36 has a first end 36a formed with or otherwise coupled to the plate 38, a second end 36b formed with or otherwise coupled to the ratchet wheel 40, and an annular outer surface 36c extending therebetween. The plate 38 and ratchet wheel 40 are laterally spaced from one another to define an annular cavity 44 configured to contain the body portion 12c of the ECG cable 12 when the ECG cable 12 is wound about the outer surface 36c of the central hub 36. The plate 38 and the central hub 36 collectively define a channel 46 through the first spool 20. The channel 46 extends diametrically through the central hub 46 while extending over/around the axle 16. The channel 46 is configured for passage of the body portion 12c of the first ECG cable 12. The central hub 36 defines a pair of recesses 48 (only one of the recesses is illustrated) extending from opposite ends of the channel 46 to resist the cable 12 from sliding out of the channel 46.

The first spool 20 is resiliently biased toward a wound condition in which the body portion 12c of the first ECG cable 12 is wound about the first spool 20 by a biasing member, such as, for example, a tension rewind spring 50. The second end 36b of the hub 36 defines a pocket (not labeled) in a lateral side thereof configured for receipt of the spring 50. The spring 50 may be retained in the pocket with a plate or cover and is attached to the axle 16. The spring 50 resiliently biases the spool 20 in a first rotational direction such that the ECG cable 14 is maintained in a wound condition about the spool 20. The ECG cable 14 may be unwound and extended from the spool 20 by applying a pulling force to the second end 12b of the ECG cable 12, thereby overcoming the resilient bias of the spring 50. A sleeve bearing 52 may be provided within a central channel 54 (FIG. 4) of the spool 20 and over the axle 16 to facilitate rotation of the spool 20 relative to the axle 16.

As best shown in FIG. 5, the ratchet wheel 40 of the spool 20 defines a plurality of notches 60 that are circumferentially spaced from one another about the ratchet wheel 40. The pawl 26 of the outer housing 12 includes a tab 62 extending out of the outer housing 12 and a tine 64 extending into the inner chamber 28 of the outer housing 12. The tine 62 is configured to selectively engage a respective notch 60 of the ratchet wheel 40 to prevent rotation of the first spool 20 in the first direction. The tab 62 may be actuated by a user (e.g., pushed down towards the base 22) to disengage the tine 62 from the notch 60 to allow the resilient bias of the spring 50 to rotate the spool 20 in the first direction back to the wound condition in which the body portion 14c of the ECG cable 14 is wrapped or coiled about the hub 36 of the spool 20.

To replace one or more of the ECG cables 14 and/or to clean the spools 20, the cover 24 may be pivoted relative to the base 20 to open the outer housing 12, as shown in FIG. 2. With the outer housing 12 opened, the axle 16, with the spools 20 attached thereto, may be detached from base 22 and removed from the outer housing 12. The ECG cable 14 positioned in an end-most spool 20 may be slid laterally out of the channel 46 of the spool 20 and then pulled through the openings 30, 32 of the base 22. The end-most spool 20 may be removed from the axle 16 to gain access to the spindle 20 positioned adjacent the end-most spindle 20 to allow for removal of the ECG cable 14 associated therewith. This process may be repeated until all of the ECG cables 14 are removed.

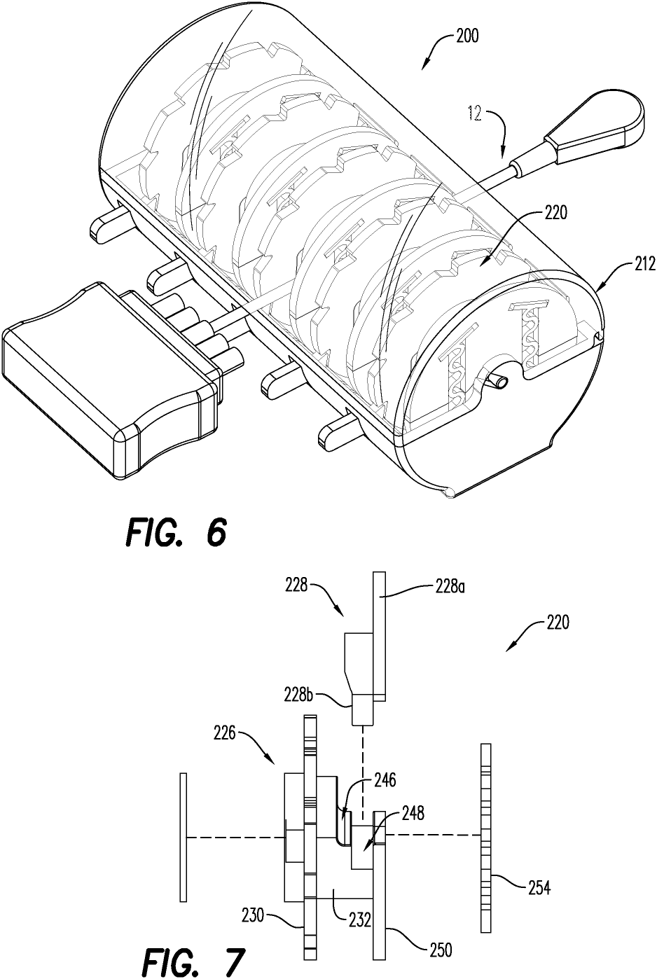

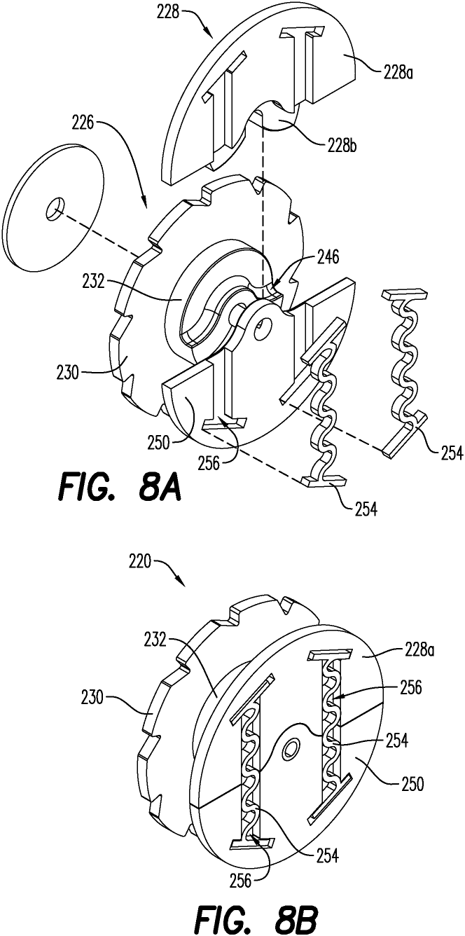

With reference to FIG. 6-8B, another embodiment of a wire organizer 200 is illustrated. The wire organizer 200 is substantially similar to the wire organizer 10 of FIGS. 1-5, and therefore only selected features of the wire organizer 200 will be described in detail herein. The wire organizer 200 includes a plurality of spools 220 rotatably supported on an axle (not labeled) and positioned within an outer housing 212. Each of the spools 20 includes a main part 226 and a secondary part 228 configured to detachably couple to the main part 226. The main part 226 includes a plate, such as, for example, a ratchet wheel 230, and a central hub 232 formed with or otherwise coupled to a central portion of the rachet wheel 230. The central hub 232 defines a channel 246 diametrically therethrough configured for passage of an ECG cable 14. The central hub 232 includes a semicircular plate 250 formed with or otherwise coupled to an end of the central hub 232. The central hub 232 further defines a groove 248 in an end thereof.

The secondary part 228 may include a semicircular plate 228a and a protrusion 228b extending from the semicircular plate 228. It is contemplated that the plates 228a, 250 may assume other suitable shapes. The protrusion 228b is configured for removable receipt in the groove 248 of the central hub 232, such that the semicircular plate 228a and the semicircular plate 250 couple to one another to collectively define a circular plate. One or more fasteners 254 may be used to retain the secondary part 228 to the main part 226. For example, the fasteners 254 may be springs received in corresponding slots 256 defined in the semicircular plates 228, 250 to retain the semicircular plate 250 with the semicircular plate 228.

With the secondary part 228 joined with the main part 226, the ECG cable 14 is prevented from being removed from the channel 246 by being captured between the protrusion 228b of the secondary part 228 and the hub 232 of the main part 226. To remove the ECG cable 14 from the spool 20, the springs 254 may be removed from the slots 256, whereby the secondary part 228 may be detached from the main part 226.

This disclosure, in various embodiments, configurations and aspects, includes components, methods, processes, systems, and/or apparatuses as depicted and described herein, including various embodiments, sub-combinations, and subsets thereof. This disclosure contemplates, in various embodiments, configurations and aspects, the actual or optional use or inclusion of, e.g., components or processes as may be well-known or understood in the art and consistent with this disclosure though not depicted and/or described herein.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

In this specification and the claims that follow, reference will be made to a number of terms that have the following meanings. The terms “a” (or “an”) and “the” refer to one or more of that entity, thereby including plural referents unless the context clearly dictates otherwise. As such, the terms “a” (or “an”), “one or more” and “at least one” can be used interchangeably herein. Furthermore, references to “one embodiment”, “some embodiments”, “an embodiment” and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Terms such as “first,” “second,” “upper,” “lower” etc. are used to identify one element from another, and unless otherwise specified are not meant to refer to a particular order or number of elements.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur - this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of.” Where necessary, ranges have been supplied, and those ranges are inclusive of all sub-ranges therebetween. It is to be expected that the appended claims should cover variations in the ranges except where this disclosure makes clear the use of a particular range in certain embodiments.

The terms “determine”, “calculate” and “compute,” and variations thereof, as used herein, are used interchangeably and include any type of methodology, process, mathematical operation or technique.

This disclosure is presented for purposes of illustration and description. This disclosure is not limited to the form or forms disclosed herein. In the Detailed Description of this disclosure, for example, various features of some exemplary embodiments are grouped together to representatively describe those and other contemplated embodiments, configurations, and aspects, to the extent that including in this disclosure a description of every potential embodiment, variant, and combination of features is not feasible. Thus, the features of the disclosed embodiments, configurations, and aspects may be combined in alternate embodiments, configurations, and aspects not expressly discussed above. For example, the features recited in the following claims lie in less than all features of a single disclosed embodiment, configuration, or aspect. Thus, the following claims are hereby incorporated into this Detailed Description, with each claim standing on its own as a separate embodiment of this disclosure.

Advances in science and technology may provide variations that are not necessarily express in the terminology of this disclosure although the claims would not necessarily exclude these variations.

What is claimed is:

1. A cable organizer comprising:
an outer housing;
an axle extending longitudinally through the outer housing; and
a plurality of spools supported on the axle such that the plurality of spools are configured to independently rotate relative to one another about a longitudinal axis defined by the axle, each spool of the plurality of spools defining a channel therethrough configured for passage of a respective electrocardiogram (ECG) cable of a plurality of ECG cables.

2. The cable organizer of claim 1, further comprising a plurality of biasing members positioned between the respective plurality of spools and the axle, wherein the plurality of biasing members are configured to rotate the plurality of spools in a first direction.

3. The cable organizer of claim 2, wherein a first spool of the plurality of spools includes a ratchet wheel defining a plurality of notches that are circumferentially spaced from one another about the ratchet wheel, the outer housing including a pawl configured to selectively engage a notch of the plurality of notches to prevent rotation of the first spool in the first direction.

4. The cable organizer of claim 3, wherein the outer housing includes:
a base; and
a cover pivotably coupled to the base, the base and the cover collectively defining an inner chamber in which the plurality of spools are received, the pawl being pivotably coupled to the base.

5. The cable organizer of claim 3, wherein the first spool includes:
a hub having a first end, a second end, and an outer surface, the ratchet wheel being coupled to the first end of the hub; and
a plate laterally spaced from the ratchet wheel and coupled to the second end of the hub, wherein a first ECG cable of the plurality of ECG cables is configured to wind about the outer surface of the hub during rotation of the first spool.

6. The cable organizer of claim 5, wherein the plate and the hub collectively define
the channel of the first spool.

7. The cable organizer of claim 1, wherein the plurality of spools is configured to slide off the axle when the axle is removed from the outer housing.

8. The cable organizer of claim 5, wherein the hub defines a pair of recesses extending from opposite ends of the channel.

9. The cable organizer of claim 1, further comprising a tension rewind spring supported in a first spool of the plurality of spools and wrapped about the axle.

10. A cable organizer comprising:
a plurality of electrocardiogram (ECG) cables, a first ECG cable of the plurality of ECG electrodes having a first end, a second end, and a body portion extending between the first and second ends;
an outer housing defining an inner chamber therein;
an axle extending longitudinally through the outer housing and supported by the outer housing; and
a plurality of spools supported on the axle, a first spool of the plurality of spools defining a channel therethrough configured for passage of the body portion of the first ECG cable, the first spool being resiliently biased toward a wound condition in which the body portion of the first ECG cable is wound about the first spool, wherein the first spool is configured to rotate from the wound condition to unwind the body portion of the first ECG cable when a pulling force is applied to the first or second end of the ECG cable.

11. The cable organizer of claim 10, wherein the first spool includes:
a main part defining the channel therethrough; and
a secondary part configured to detachably couple to the main part, wherein the main part and the secondary part, when coupled to one another, are configured to capture the body portion of the first ECG cable therebetween.

12. The cable organizer of claim 11, wherein the first spool further includes at least one fastener configured to detachably secure the secondary part to the main part.

13. The cable organizer of claim 11, wherein the main part includes:
a central hub having a first end and a second end;
a ratchet wheel formed with the first end of the central hub; and
a first semicircular plate formed with the second end of the central hub, and wherein the secondary part includes:
a second semicircular plate configured to join with the first semicircular plate of the main part; and
a protrusion extending from the second semicircular plate of the secondary part and configured to couple to the central hub, whereby the central hub and the protrusion capture the body portion of the first ECG cable therebetween.

14. The cable organizer of claim 10, further comprising a plurality of biasing members positioned between the respective plurality of spools and the axle, wherein the plurality of biasing members is configured to rotate the plurality of spools in a first direction toward the wound condition.

15. The cable organizer of claim 14, wherein the first spool includes a ratchet wheel defining a plurality of notches that are circumferentially spaced from one another about the ratchet wheel, the outer housing including a pawl configured to selectively engage a respective notch of the plurality of notches to prevent rotation of the first spool in the first direction.

16. The cable organizer of claim 15, wherein the first spool includes:
a hub having a first end, a second end, and an outer surface, the ratchet wheel being coupled to the first end of the hub; and
a plate laterally spaced from the ratchet wheel and coupled to the second end of the hub, the first ECG cable being configured to wind about the outer surface of the hub during rotation of the first spool in the first direction.

17. The cable organizer of claim 16, wherein the plate and the hub collectively define the channel of the first spool.

18. The cable organizer of claim 16, wherein the hub defines a pair of recesses extending from opposite ends of the channel.

19. The cable organizer of claim 10, wherein the outer housing includes:
a base; and
a cover pivotably coupled to the base, the base and the cover collectively defining the inner chamber of the outer housing.

20. The cable organizer of claim 10, further comprising a tension rewind spring supported in the first spool and wrapped about the axle.
